(19) United States
(12) Patent Application Publication
(10) Pub. No.: US 2006/0079568 A1
(43) Pub. Date: Apr. 13, 2006

(54) Inhibition of atherosclerosis by diindolylmethane analogs

(75) Inventors: Stephen H. Safe, Bryan, TX (US); Ismael J. Samudio, Houston, TX (US); Paolo Calabro, Naples (IT); Edward T.H. Yeh, Houston, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: The Texas A&M University System, College Station, TX (US)

(21) Appl. No.: 11/133,679

(22) Filed: May 20, 2005

Related U.S. Application Data
(60) Provisional application No. 60/573,535, filed on May 21, 2004.

(51) Int. Cl. A61K 31/405

(52) U.S. Cl. 514/414

(57) ABSTRACT

Diindolylmethane analogs such as 1,1-bis(3′-indolyl)-1-(p-substituted phenyl)methanes can be used to treat atherosclerosis and other vascular disease states. The analogs have been shown to display antiinflammatory effects in endothelial cells, suggesting their clinical applicability.

This application claims the benefit under 35 U.S.C. 119(e) of U.S. Provisional Application Ser. No. 60/573,535, filed May 21, 2004, the entire contents of which are incorporated herein by reference.

### STATEMENT REGARDING FEDERALLY SPONSORED RESEARCH OR DEVELOPMENT

The government may own rights in the present invention pursuant to grant number ESO-9106 from the National Institute of Health and the DREAMS (Disaster Relief and Emergency Medical Services) project from the U.S. Department of the Army.

### FIELD OF THE INVENTION

The invention relates to the treatment of atherosclerosis and heart disease using diindolylmethane analogs.

### DESCRIPTION OF RELATED ART

The adhesion of leukocytes to vascular endothelial cells is a critical step in the development of atherosclerosis and involves the recruitment of leukocytes to the site of tissue injury or lesion formation and their infiltration into the vessel wall. There are several cytokines involved in this process.

One important cytokine in this process is the intercellular adhesion molecule-1 (ICAM)-1, which is expressed on endothelial cells. It is one of the major cell surface glycoproteins that promote cell adhesion [1]. Although ICAM-1 is constitutively expressed in endothelial cells, its levels can be significantly raised in response to proinflammatory mediators, such as tumor necrosis factor-α (TNF-α) [2], which may further contribute to the role of ICAM-1 in atherosclerosis. Specific chemokines, particularly monocyte chemoattractant protein-1 (MCP-1) and interleukin 6 (IL-6), which are also expressed by endothelial cells, have a major role in the development of atherosclerosis as well.

Another important cytokine in the pathogenesis of atherosclerosis is peroxisome proliferator-activated receptor-γ (PPAR-γ), a ligand-activated nuclear receptor that has an essential role in adipogenesis and glucose homeostasis and is expressed in atherosclerotic plaques [3]. PPAR-γ is also expressed in vessel wall tissues, including endothelial cells (ECs) [4]. Although the role of PPAR-γ in inflammation, and in particular its role in the activation of ECs, is unclear, it is possible that ligand-dependent activation of PPAR-γ might constitute an effective strategy for managing atherosclerosis.

Recently we studied the mode of action of 1,1-bis(3′-indolyl)-1-(p-trifluoromethylphenyl) methane (DIM-C-pPhCF₃) and other p-substituted phenyl DIM analogs, which constitute a new class of PPAR-γ agonists that resemble the natural ligand 15-deoxy-δ^{12,14}-prostaglandin J2 (15d-PGJ2), in MCF-7 breast and other cancer cells [5]. However, given the possible role of PPAR-γ in the pathogenesis of atherosclerosis, we hypothesized that PPAR-γ agonists might also be effective in opposing the inflammation associated with atherosclerosis.

### SUMMARY OF THE INVENTION

Diindolylmethane analogs are effective to inhibit vascular inflammation. One or more analogs can be used in the treatment of atherosclerosis and related vascular problems.

### DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1 shows the effects of three members of the new class of PPAR-γ agonists on the TNF-α-induced expression of ICAM-1 in HUVECs. Cells were pretreated with DIM-C-pPhtBu (A), DIM-C-pPhC₆H₅ (B), or DIM-C-pPhCH₃ (C) at the concentrations shown for 6 hours and then incubated with 5 ng/ml TNF-α for 24 hours. Cell surface expression of ICAM-1 was measured by FACS. Data are expressed as the mean ±SD of a representative experiment performed in triplicate. *P<0.05.

FIG. 2 shows a comparison of the effects of different PPAR-γ agonists on TNF-α-induced expression of ICAM-1 in HUVECs. Cells were pretreated with 10 μmol/L DIM-C-pPhCH₃, DIM-C-pPhtBu, DIM-C-pPhC₆H₅, 15d-PGJ2, or ciglitazone for 6 hours and then incubated for 24 hours with 5 ng/ml TNF-α. Cell surface expression of ICAM-1 was measured by FACS. Data are expressed as the mean ±SD of a representative experiment performed in triplicate. *P<0.05.

FIG. 3 shows the effects of different PPAR-γ agonists on IL-6 production in HUVECs stimulated with TNF-α. HUVECs were seeded in 24-well plates. After 2 days, the cells were first pretreated with different PPAR-γ agonists at a dose of 10 μmol/L for 6 hours and then incubated with 5 ng/ml TNF-α for 24 hours. IL-6 concentrations in the culture supernatants were measured by ELISA. Data are expressed as the mean ±SD of a representative experiment performed in triplicate. *P<0.05.

FIG. 4 shows the effects of different PPAR-γ agonists on MCP-1 production in HUVECs stimulated with TNF-α. HUVECs were seeded in 24-well plates. After 2 days, the cells were first pretreated with different PPAR-γ agonists at a dose of 10 μmol/L for 6 hours and then incubated with 5 ng/ml TNF-α for 24 hours. MCP-1 concentrations in the culture supernatants were measured by ELISA. Data are expressed as the mean ±SD of a representative experiment performed in triplicate. *P<0.05.

### DETAILED DESCRIPTION OF THE INVENTION

The effects of this new class of PPAR-γ agonists on vascular inflammation were assessed by investigating the expression of selected chemokines, such as IL-6, MCP-1, and ICAM-1, following EC activation by TNF-α.

ECs are primary cellular targets for the actions of proinflammatory cytokines, such as TNF-α, which are produced predominantly by activated macrophages [6]. The binding of TNF-α to the p55 TNF receptor may lead to EC activation. The TNFα-mediated inflammatory response involves the induction of cell adhesion molecules, including ICAM-1 (CD54) and VCAM-1 (CD106) [7,8]. The interaction of inflammatory cells with other cells via ICAM and VCAM is a necessary first step in atherogenesis [9]. Once they adhere to the endothelium, inflammatory cells migrate into the subendothelial space, attracted by MCP-1 [10].

In response to several atherogenic stimulants such as oxidized low-density lipoprotein and interleukin (IL)-1, MCP-1 is induced in endothelial cells and promotes the transmigration of monocytes through the endothelial barrier, which is thought to be the earliest and most significant event in the formation of atherosclerotic lesions [11,12]. A major role for MCP-1 in atherogenesis is supported by the observation that disruption of the MCP-1 gene markedly reduced the development of atherosclerosis in low-density-lipoprotein receptor-deficient or apolipoprotein B-overexpressing mice [10,13]. IL-6 is a circulating cytokine secreted by numerous different cells, including activated macrophages, lymphocytes, and endothelial cells. It might therefore play a key role in the development of coronary disease through a number of different mechanisms [14].

PPAR-γ is a member of the nuclear receptor superfamily of ligand-activated transcription factors [15-17]. PPAR-γ is highly expressed in tumors and cancer cell lines, and agonists for this receptor inhibit tumor growth [5,18-20]. PPAR-γ is also highly expressed in adipose tissue and in other tissues, including endothelial cells [4]. Further, PPAR-γ has been identified in atherosclerotic plaques, and the ligand-dependent activation of PPAR-γ inhibits monocyte activation [21].

Previous studies by the inventors and others have shown that PPAR-γ agonists, such as 15d-PGJ2 and the thiazolidinedione (TZD) class of insulin-sensitizing drugs can modulate the expression of many pro-inflammatory cytokines [3,21], chemokines [22], and adhesion molecules [23] in macrophages and other cell types, including ECs. These effects result from the targeting of multiple pathways and include inhibition of NFκB-dependent responses [24]. Interactions between the PPAR-γ and NFκB signaling pathways result in the downregulation of proteins involved in the inflammatory process. However, some studies [25,26] have not shown modulation of the inflammatory process by PPAR-γ agonists, and this may be due, in part, to the variable doses and structures of PPAR-γ agonists used in these studies.

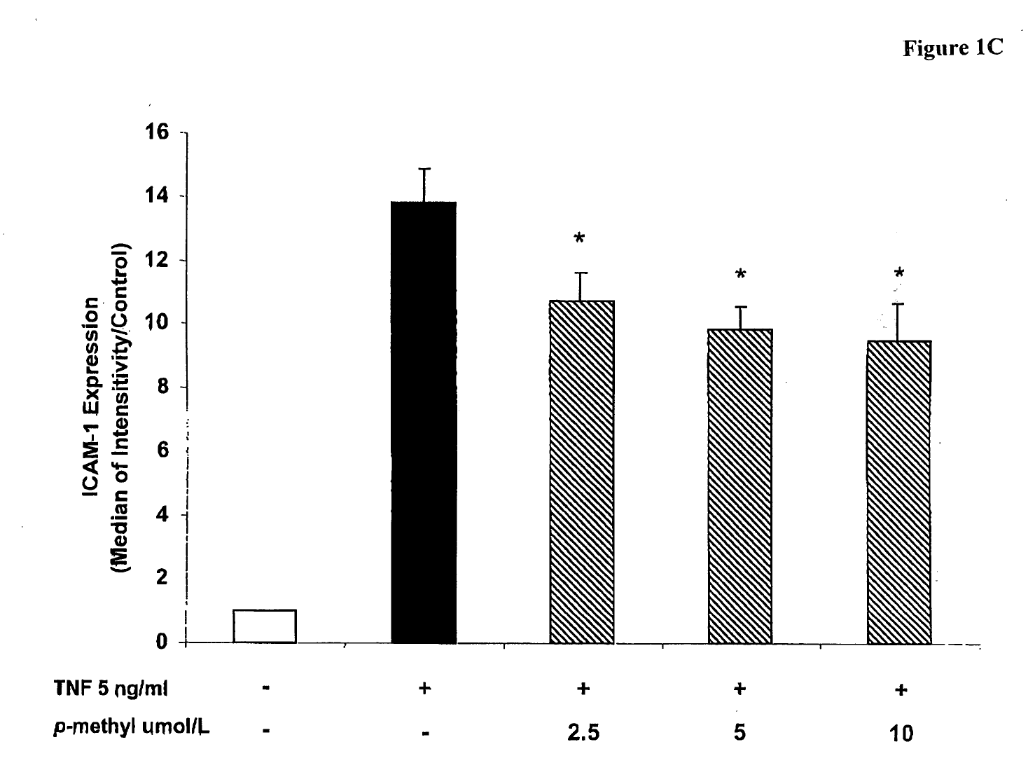

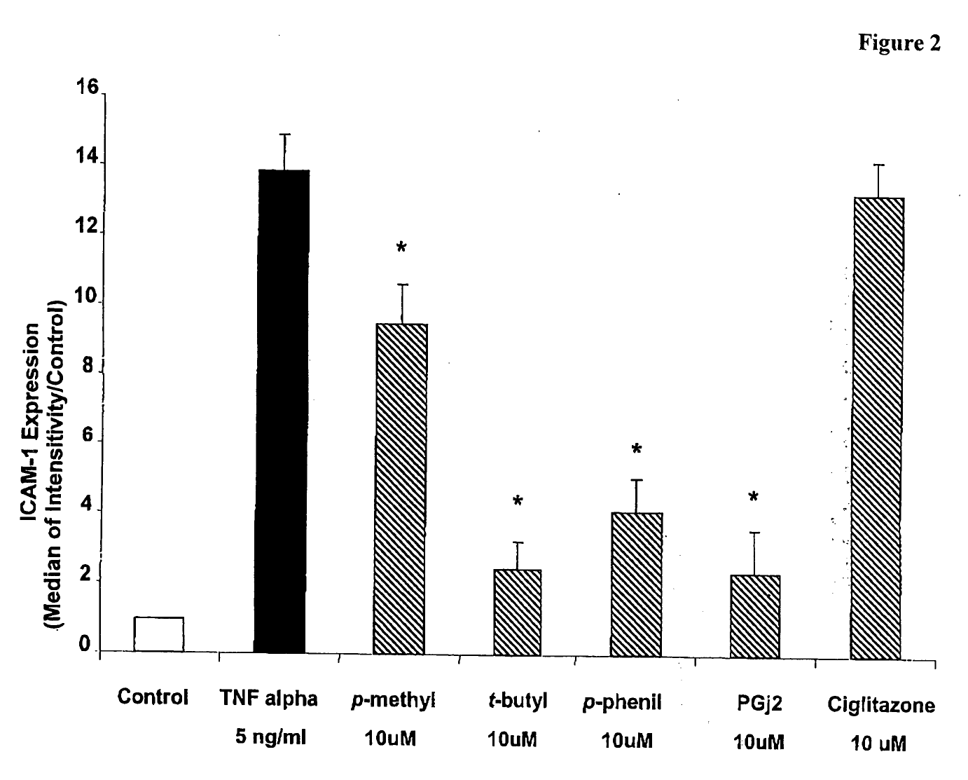

15d-PGJ2 and the TZDs represent two important classes of PPAR-γ agonists, and previous studies in our laboratory have shown that PPAR-γ activators markedly decrease the expression of adhesion molecules in activated human ECs. Moreover, short-term treatment with the PPAR-γ agonist, troglitazone, significantly inhibited macrophage homing to atherosclerotic plaques [23]. FIG. 2 demonstrates that 10 μmol/L 15d-PGJ2 significantly inhibited TNF-α-induced ICAM-1 expression and IL-6 and MCP-1 secretion in ECs, whereas ciglitazone was inactive at this concentration.

This application discloses the use of a new class of PPAR-γ agonists as inhibitors of TNF-α-induced responses in ECs and compared their potencies to 15d-PGJ2 and ciglitazone. The compounds selected for this study consisted of two potent (DIM-C-pPhtBu and DIM-C-pPhC₆H₅) and one less active (DIM-C-pPhCH₃) analog, as demonstrated in previous structure-activity relationship studies in cancer cell lines [5].

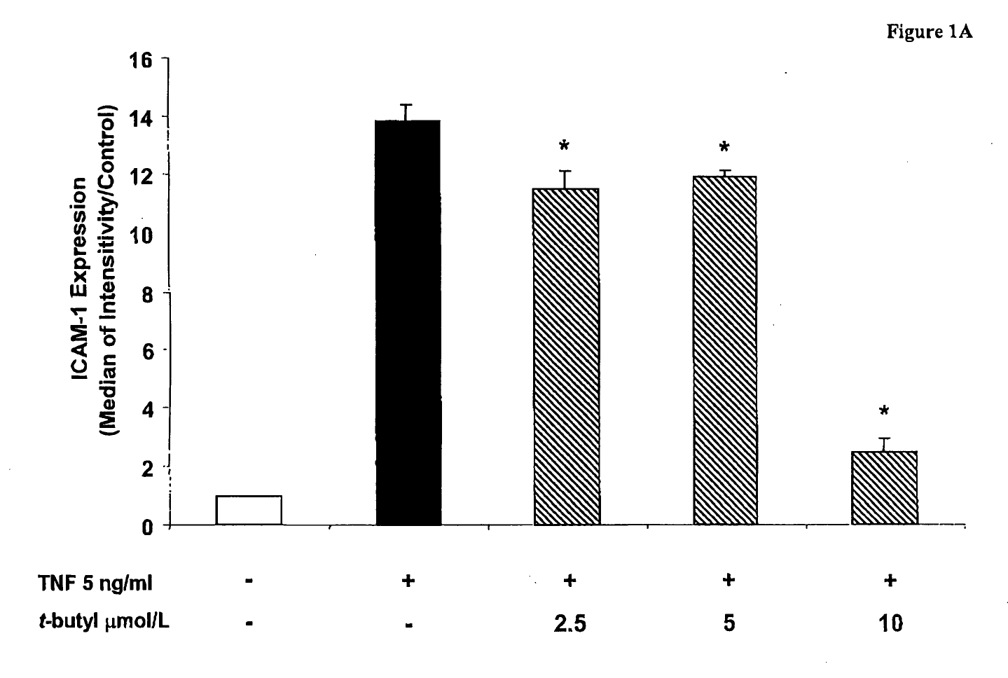

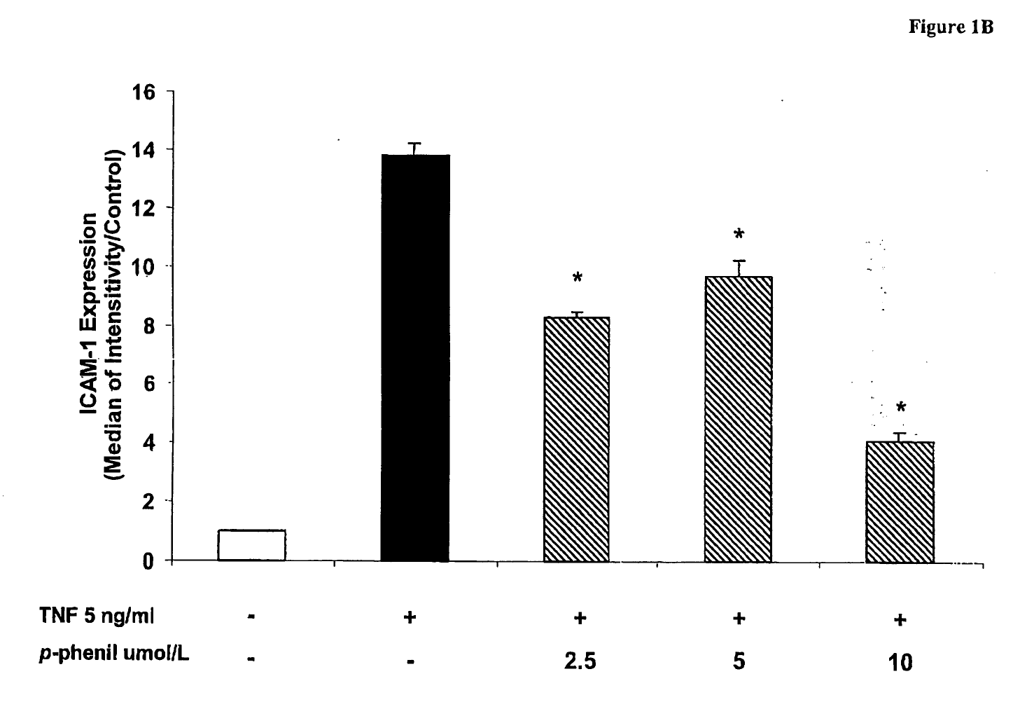

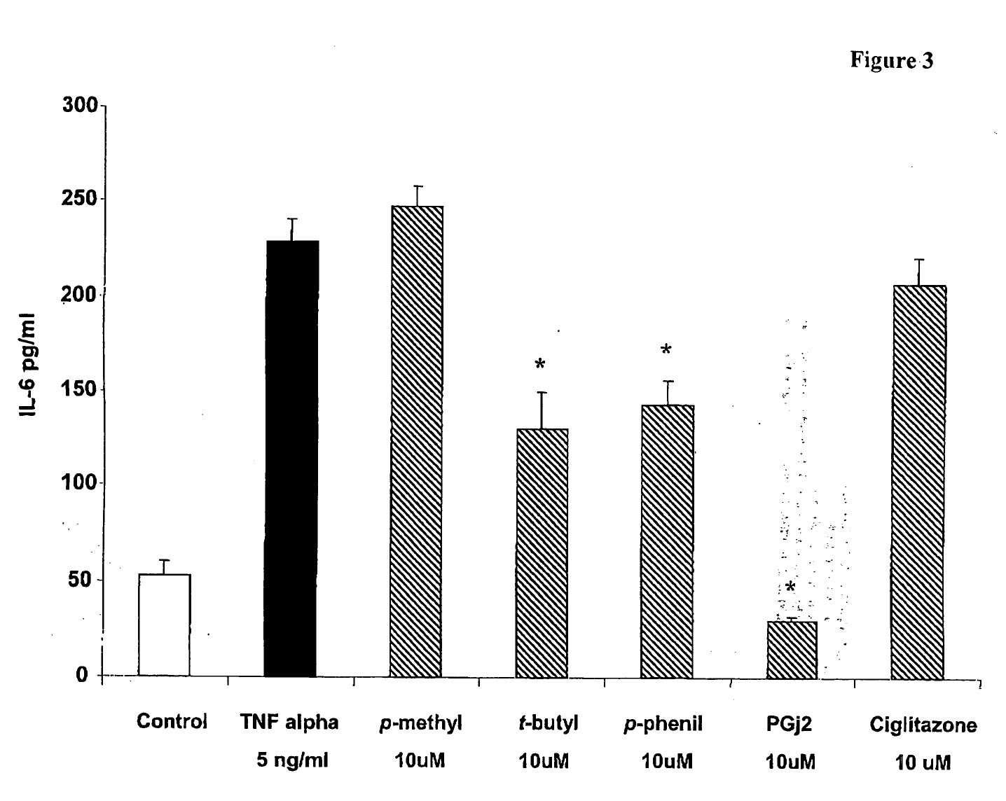

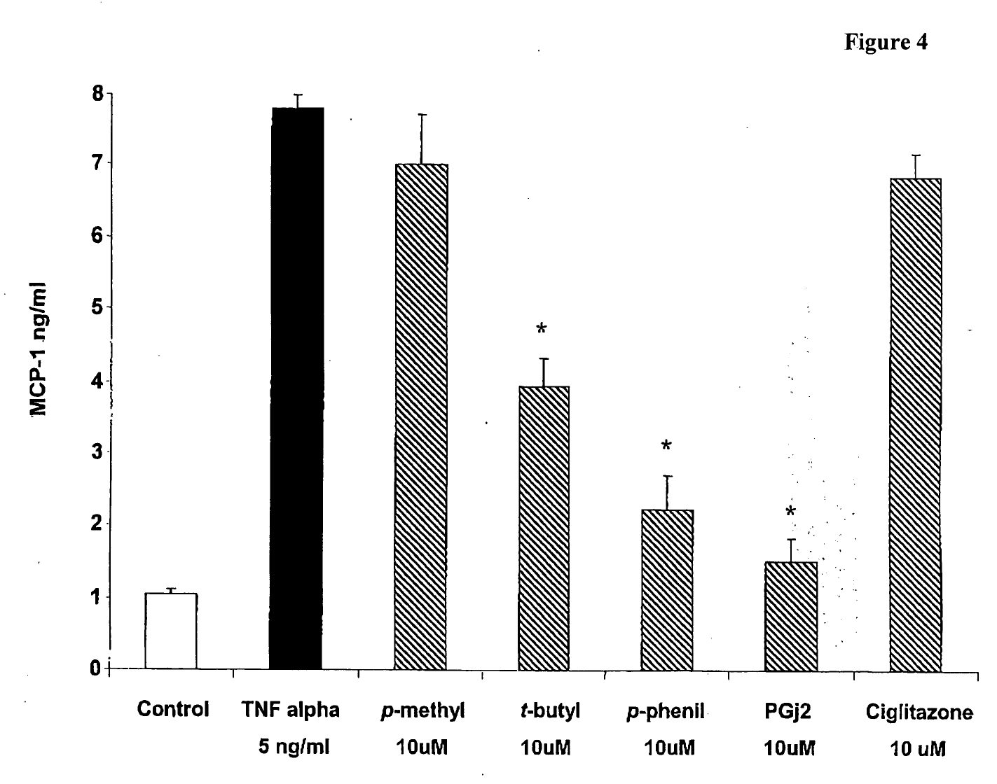

The instant inventors found that both DIM-C-pPhtBu and DIM-C-pPhC₆H₅ inhibited TNF-α-induced ICAM-1 expression (FIGS. 1A and B) and IL-6 and MCP-1 production (FIGS. 3 and 4) in ECs and that their potencies were comparable to those of 15d-PGJ2. In contrast, DIM-C-pPhCH₃ (FIGS. 1C, 3, and 4) exhibited lower activity, which is consistent with the observations made in structure-activity studies of these compounds [5]. The DIM analogs are well tolerated in animal studies [5,27-29], and this, together with their relatively potent ability to inhibit atherosclerotic processes, suggests that these PPAR-γ agonists hold promise for the treatment of endothelial inflammatory processes.

Proinflammatory cytokines and adhesion molecules expressed by endothelial cells play a critical role in initiating and promoting atherosclerosis. Agents that oppose these inflammatory effects in vascular cells include peroxisome proliferator-activated receptor-γ (PPAR-γ) ligands, including 15-deoxy-δ^{12,14}-prostaglandin J2 (15d-PGJ2) and synthetic thiazolidinediones. Recently, a new structural class of potent PPAR-γ agonists, 1,1-bis(3′-indolyl)-1-(p-substituted phenyl) methanes, has been characterized. The purpose of the present study was to evaluate the antiinflammatory effects of two active members of this class, 1,1-bis(3′-indolyl)-1-( p-t-butylphenyl) methane (DIM-C-pPhtBu) and 1,1-bis(3′-indolyl)-1-( p-biphenyl) methane (DIM-C-pPhC₆H₅), in endothelial cells in vitro.

Pretreatment of endothelial cells with DIM-C-pPhC₆H₅, DIM-C-pPhtBu, or 15d-PGJ2 decreased tumor necrosis factor-α (TNF-α)-induced intercellular adhesion molecule (ICAM)-1 expression in a concentration-dependent manner. Specifically, at a concentration of 10 μmol/L, DIM-C-pPhtBu and DIM-C-pPhC₆H₅ decreased ICAM-1 expression by 77.5% and 71.3%, respectively, from that induced in control cells. A significant inhibition (84.4%) was also seen for 10 μM 15d-PGJ2 (P<0.05). In contrast, ciglitazone and DIM-C-pPhCH₃ which have low PPAR-γ agonist activity, were inactive at 10 μM. The two new PPAR-γ agonists and 15d-PGJ2 also inhibited TNF-α-induced interleukin 6 and monocyte chemoattractant protein-1 production in supernatants of TNF-α-stimulated endothelial cells. Ciglitazone and DIM-C-pPhCH₃ did not decrease TNF-α-induced expression of these two proteins.

This structural class of PPAR-γ agonists inhibited the expression of ICAM-1 and the production of interleukin 6 and monocyte chemoattractant protein-1 in TNF-α-activated endothelial cells at lower concentrations than those of other synthetic PPAR-γ agonists required to achieve the same effect. These results indicate the potential clinical usefulness of 1,1-bis(3′-indolyl)-1-(p-substituted phenyl) methanes in the reduction of endothelial inflammation.

One embodiment of the invention includes the treatment of atherosclerosis or other heart disease by the administration of diindolylmethane analogs. The treatment can generally be performed in any mammal. Examples of mammals includes humans, dogs, cats, cows, horses, pigs, goats, bears, moose, and so on. It is presently preferred that the mammal be a human. The administration can generally be performed by any method suitable to deliver the diindolylmethane analog to an appropriate site in the body. Administration can include injection (such as IV, IP, or IM), oral, intranasal, transdermal, or other methods.

The treatment method can generally comprise selecting a patient diagnosed with or suspected of having atherosclerosis, and administering a formulation comprising a diindolylmethane analog.

Diindolylmethane analogs have been disclosed in U.S. Pat. No. 5,948,808 (issued Sep. 7, 1999) and U.S. Patent Publication No. 2002-0115708-A1 (Aug. 22, 2002). The analogs can include 1,1-bis(3′-indolyl)-1-(p-substituted phenyl)methanes. Two specific examples include 1,1-bis(3′-indolyl)-1-( p-t-butylphenyl) methane (DIM-C-pPhtBu) and 1,1-bis(3′-indolyl)-1-( p-biphenyl) methane (DIM-C-pPhC₆H₅).

The diindolylmethane analog can be formulated as a liquid solution in water or other solvent, or as a solid such as a pill, tablet, capsule, or powder. The concentration of analog in the formulation can generally be any concentration suitable for treating atherosclerosis or other heart disease. The formulation can comprise one or more diindolylmethane analogs. The formulation can also comprise other materials such as binders, fillers, colorants, solvents, surfactants, or other bioactive materials.

The treatment of atherosclerosis or other heart disease preferably reduces or eliminates the presence or symptoms of the condition. The reduction is preferably at least about 10%, at least about 20%, at least about 30%, at least about 40%, at least about 50%, at least about 60%, at least about 70%, at least about 80%, at least about 90%, and ideally 100%.

Administration of the formulation can be performed in a single dose, multiple doses, or as a continual administration. Administration time and concentration can be varied during the treatment depending on the observed effects of the treatment.

The diindolylmethane analog can also be used in methods to reduce expression of tumor necrosis factor-α (TNF-α-induced intercellular adhesion molecule (ICAM)-1, TNF-α-induced interleukin 6, and monocyte chemoattractant protein-1.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Chemical and Cell Culture

Human umbilical vein ECs (HUVECs, Cascade Biology, Portland, Oreg.) were grown in M199 medium (GIBCO, Carlsbad, Calif.) with 15% fetal bovine serum (Sigma Chemical Co., St. Louis, Mo.), 0.2 mg/ml heparin, 0.1 mg/ml EC growth supplement (Biomedical Technologies, Stoughton, Mass.), 2 mmol/L L-glutamine, and 1% penicillin/streptomycin. Cells from passages 2 to 4 were used in the experiments. The p-substituted phenyl DIM analogs containing p-t-butyl (DIM-C-pPhtBu), p-phenyl (DIM-C-pPhC₆H₅), and p-methyl (DIM-C-pPhCH₃) substituents used in the study were >95% pure and were prepared by the condensation of indole with the corresponding p-substituted benzaldehydes. DIM-C-pPhC₆H₅ and DIM-C-pPhtBu are active agents, as shown by earlier structure-activity studies, whereas DIM-C-pPhCH₃ is a relatively inactive PPARy agonist [5].

### Example 2

### Detection of ICAM-1

The expression of ICAM-1 on the cell surface was determined in HUVECs cultured in six-well plates pretreated with one of the three different p-substituted phenyl DIM analogs or with vehicle (0.1% DMSO) at the concentrations indicated. Their effects were compared with those of other PPARγ agonists by preincubating HUVECs with ciglitazone (Biomol, Plymouth meeting, Pa.) or 15d-PGJ2 (Calbiochem, San Diego, Calif.) at the same doses.

After 6 hours, cells were incubated with TNF-α (R&D Systems, Minneapolis, Minn.) at a concentration of 5 ng/ml for 12 hours. Cells were then detached with 10 mmol/L EDTA in 0.5% phosphate-buffered saline, collected by centrifugation, and stained for 30 minutes on ice in the dark with R-phycoerythrin-labeled monoclonal antibody against ICAM-1 (CD54) or with the appropriate R-phycoerythrin-labeled isotype IgG (Pharmingen, San Diego, Calif.) as a control.

The fluorescence intensity of 10,000 gated viable cells was analyzed for each sample on a FACSCalibur Flow Cytometer (Becton Dickinson Immunocytometry Systems, San Diego, Calif.) using Cell Quest (Becton Dickinson) acquisition software. All experiments were performed in triplicate.

### Example 3

### Chemokine Assays

In order to measure chemokine levels in the cell supernatant, HUVECs cultured in 24-well plates were preincubated for 6 hours with one of the three p-substituted phenyl DIM analogs at the concentrations indicated or with vehicle and then stimulated with 5 ng/ml TNF-α. For comparison, HUVECs were also preincubated with ciglitazone or 15d-PGJ2 at the same concentrations and then stimulated with TNF-α at a concentration of 5 ng/ml. Cell culture supernatants were collected 6 and 24 hours after the stimulation for analysis of IL-6 and MCP-1, respectively.

The levels of IL-6 and MCP-1 were quantified using commercial ELISA kits (BioSource International, Camarillo, Calif.) according to the manufacturer's directions. The minimum detectable concentration of the assay was 2 pg/ml for IL-6 and <20 pg/ml for MCP-1. All experiments were performed in triplicate.

### Example 4

### Statistical Analysis

Data are reported as means±standard deviation. Differences were analyzed by ANOVA followed by the Fisher least significant difference test. A P value of <0.05 was considered significant.

We therefore assessed the effects of this new class of PPAR-γ agonists on vascular inflammation by investigating the expression of the chemokines IL-6, MCP-1, and ICAM-1 following EC activation by TNF-α.

### Example 5

### Effect of p-substituted Phenyl DIM Analogs on ICAM-1 Expression in HUVECs

HUVECs expressed low basal levels of ICAM-1. Similarly, treatment with different concentrations (up to 10 μmol/L) of one of the three p-substituted phenyl DIM analogs, with ciglitazone, or 15d-PGJ2 did not induce apoptosis or change the baseline expression of ICAM-1 (data not shown). In contrast, incubation of HUVECs with TNF-α 5 ng/ml for 12 hours significantly increased the expression of ICAM-1. Conversely, pretreatment of HUVECs with DIM-C-pPhtBu (FIG. 1A) decreased the expression of ICAM-1 in a concentration-dependent manner. In particular, 10 μmol/L DIM-C-pPhtBu maximally reduced the expression of ICAM-1 by 77.5%. DIM-C-pPhC₆H₅ had a similar effect (FIG. 1B), with a maximal reduction in ICAM-1 expression of 71.3% observed for a dose of 10 μmol/L (P<0.05). However, pretreatment with 10 μmol/L DIM-C-pPhCH₃ (FIG. 1C) induced only a small, but significant 32% decrease in the expression of TNFα-induced ICAM-1. This order of potency--DIM-C-pPhtBu≅DIM-C-pPhC₆H₅>DIM-C-pPhCH₃ parallels the relative PPAR-γ agonist activities of these compounds observed in transactivation assays [5].

On the basis of these results, we chose 10 μmol/L as the concentration for the comparison experiments examining other PPAR-γ agonists. These experiments showed that pretreatment with 15d-PGJ2 was associated with a significant (i.e., 84.4%) reduction in ICAM-1 expression compared with the untreated TNF-α-stimulated HUVECs. However, pretreatment with 10 μmol/L ciglitazone had no inhibitory effect on TNFα-induced ICAM-1 expression in HUVECs (FIG. 2).

### Example 6

### Effects of PPAR-γ Agonists on Production of IL-6 and MCP-1 by TNF-α-Stimulated HUVECs Chemical and Cell Culture

To determine the effects of the three p-substituted phenyl DIM analogs on TNF-α-induced chemokine production in HUVECs, cells were pretreated for 6 hours with one of the three analogs at the concentrations indicated or with vehicle and then stimulated with 5 ng/ml TNF-α for the indicated time before the chemokine assays were performed.

As expected, the levels of IL-6 markedly increased (>4-fold) in response to TNF-α stimulation for 6 hours (from 52.8±7.5 pg/ml at baseline to 228±12.7 pg/ml, P<0.05) (FIG. 3). In contrast, the pretreatment of cells with 10 μmol/L DIM-C-pPhtBu or DIM-C-pPhC₆H₅ inhibited TNF-α-induced IL-6 production, with IL-6 levels of 130.3±19.3 pg/ml and 143.4±12.2 pg/ml, respectively, in the treatment groups. Pretreatment with DIM-C-pPhCH₃ did not significantly inhibit TNF-α-induced IL-6 production.

A similar pattern was observed in the production of MCP-1 by HUVECs. Specifically, treatment of these cells with TNF-α for 24 hours significantly induced (>7-fold) MCP-1 production (from 1.05±0.07 ng/ml at baseline to 7.8±0.19 ng/ml, P<0.05) (FIG. 4). However, the pretreatment of cells with 10 μmol/L DIM-C-pPhtBu resulted in a significant inhibition of TNF-α-induced MCP-1 production to 3.9±0.41 ng/ml (P<0.05). DIM-C-pPhC₆H₅ also strongly inhibited the TNF-α-induced production of MCP-1 in HUVECs. Specifically, MCP-1 levels were decreased to 2.2±0.49 ng/ml, whereas DIM-C-pPhCH₃, a relatively inactive PPARγ agonist, did not affect the TNF-α-induced levels of MCP-1.

In order to compare the effects of these PPAR-γ agonists with those of other known PPAR-γ agonists, HUVECs were pretreated for 6 hours with 10 μmol/L 15d-PGJ2 or ciglitazone and then stimulated with 5 ng/mL TNF-α for the indicated times before the chemokine assays were performed. 15d-PGJ2 significantly (P<0.05) inhibited TNF-α-induced IL-6 production (to 29.8±1.6 pg/ml), whereas ciglitazone only weakly affected IL-6 production (to 207±13 pg/ml, P=0.17) (FIG. 3). TNF-α-induced MCP-1 production in HUVECs was also significantly (P<0.05) inhibited after cells were pretreated with 15d-PGJ2 (to 1.5±0.3 pg/ml), whereas ciglitazone only slightly inhibited MCP-1 synthesis in HUVECs (to 6.8±0.2 pg/ml, P=0.01) (FIG. 4).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

### REFERENCES

1. Bevilacqua M P. Endothelial-leukocyte adhesion molecules. Annu Rev Immunol 1993;11:767-804.
2. Pober J S. Effects of tumor necrosis factor and related cytokines on vascular endothelial cells. Ciba Found Symp 1987; 131:170-84.
3. Ricote M, Li A C, Willson T M, Kelly C J, Glass C K. The peroxisome proliferator-activated receptor-gamma is a negative regulator of macrophage activation. Nature 1998;391:79-82.
4. Marx N, Bourcier T, Sukhova G K, Libby P, Plutzky J. PPARgamma activation in human endothelial cells increases plasminogen activator inhibitor type-1 expression: PPARgamma as a potential mediator in vascular disease. Arterioscler Thromb Vasc Biol 1999; 19:546-51.
5. Qin C, Morrow D, Stewart J, Spencer K, Porter W, Smith R, 3rd, Phillips T, Abdelrahim M, Samudio I, Safe S. A new class of peroxisome proliferator-activated receptor gamma (PPARgamma) agonists that inhibit growth of breast cancer cells: 1,1-Bis(3′-indolyl)-1-(p-substituted phenyl)methanes. Mol Cancer Ther 2004;3:247-60.
6. Locksley R M, Killeen N, Lenardo M J. The TNF and TNF receptor superfamilies: integrating mammalian biology. Cell 2001;104:487-501.
7. van de Stolpe A, van der Saag P T. Intercellular adhesion molecule-1. J Mol Med 1996;74:13-33.
8. Carter R A, Wicks I P. Vascular cell adhesion molecule 1 (CD106): a multifaceted regulator of joint inflammation. Arthritis Rheum 2001;44:985-94.
9. Adams D H, Shaw S. Leucocyte-endothelial interactions and regulation of leucocyte migration. Lancet 1994;343:831-6.
10. Gu L, Okada Y, Clinton S K, Gerard C, Sukhova G K, Libby P, Rollins B J. Absence of monocyte chemoattractant protein-1 reduces atherosclerosis in low density lipoprotein receptor-deficient mice. Mol Cell 1998;2:275-81.
11. Sasayama S, Okada M, Matsumori A. Chemokines and cardiovascular diseases. Cardiovasc Res 2000;45:267-9.
12. Glass C K, Witztum J L. Atherosclerosis. the road ahead. Cell 2001;104:503-16.
13. Gosling J, Slaymaker S, Gu L, Tseng S, Zlot C H, Young S G, Rollins B J, Charo I F. MCP-1 deficiency reduces susceptibility to atherosclerosis in mice that overexpress human apolipoprotein B. J Clin Invest 1999;103:773-8.
14. Yudkin J S, Kumari M, Humphries S E, Mohamed-Ali V. Inflammation, obesity, stress and coronary heart disease: is interleukin-6 the link? Atherosclerosis 2000;148:209-14.
15. Issemann I, Green S. Activation of a member of the steroid hormone receptor superfamily by peroxisome proliferators. Nature 1990;347:645-50.
16. Mangelsdorf D J, Thummel C, Beato M, Herrlich P, Schutz G, Umesono K, Blumberg B, Kastner P, Mark M, Chambon P, et al. The nuclear receptor superfamily: the second decade. Cell 1995;83:835-9.
17. Lemberger T, Desvergne B, Wahli W. Peroxisome proliferator-activated receptors: a nuclear receptor signaling pathway in lipid physiology. Annu Rev Cell Dev Biol 1996;12:335-63.
18. Murphy G J, Holder J C. PPAR-gamma agonists: therapeutic role in diabetes, inflammation and cancer. Trends Pharmacol Sci 2000;21:469-74.
19. Theocharisa S, Margeli A, Kouraklis G. Peroxisome proliferator activated receptor-gamma ligands as potent antineoplastic agents. Curr Med Chem Anti-Canc Agents 2003;3:239-51.
20. Keshamouni V G, Reddy R C, Arenberg D A, Joel B, Thannickal V J, Kalemkerian G P, Standiford T J. Peroxisome proliferator-activated receptor-gamma activation inhibits tumor progression in non-small-cell lung cancer. Oncogene 2004;23:100-8.
21. Jiang C, Ting A T, Seed B. PPAR-gamma agonists inhibit production of monocyte inflammatory cytokines. Nature 1998;391:82-6.
22. Marx N, Mach F, Sauty A, Leung J H, Sarafi M N, Ransohoff R M, Libby P, Plutzky J, Luster A D. Peroxisome proliferator-activated receptor-gamma activators inhibit IFN-gamma-induced expression of the T cell-active CXC chemokines IP-10, Mig, and I-TAC in human endothelial cells. J Immunol 2000;164:6503-8.
23. Pasceri V, Wu H D, Willerson J T, Yeh E T. Modulation of vascular inflammation in vitro and in vivo by peroxisome proliferator-activated receptor-gamma activators. Circulation 2000;101:235-8.
24. Straus D S, Pascual G, Li M, Welch J S, Ricote M, Hsiang C H, Sengchanthalangsy L L, Ghosh G, Glass C K. 15-deoxy-delta 12,14-prostaglandin J2 inhibits multiple steps in the NF-kappa B signaling pathway. Proc Natl Acad Sci U S A 2000;97:4844-9.
25. Thieringer R, Fenyk-Melody J E, Le Grand C B, Shelton B A, Detmers P A, Somers E P, Carbin L, Moller D E, Wright S D, Berger J. Activation of peroxisome proliferator-activated receptor gamma does not inhibit IL-6 or TNF-alpha responses of macrophages to lipopolysaccharide in vitro or in vivo. J Immunol 2000;164:1046-54.
26. Moore K J, Rosen E D, Fitzgerald M L, Randow F, Andersson L P, Altshuler D, Milstone D S, Mortensen R M, Spiegelman B M, Freeman M W. The role of PPAR-gamma in macrophage differentiation and cholesterol uptake. Nat Med 2001;7:41-7.
27. Chen I, McDougal A, Wang F, Safe S. Aryl hydrocarbon receptor-mediated antiestrogenic and antitumorigenic activity of diindolylmethane. Carcinogenesis 1998;19:1631-9.
28. McDougal A, Sethi Gupta M, Ramamoorthy K, Sun G, Safe S H. Inhibition of carcinogen-induced rat mammary tumor growth and other estrogen-dependent responses by symmetrical dihalo-substituted analogs of diindolylmethane. Cancer Lett 2000;151:169-79.
29. McDougal A, Gupta M S, Morrow D, Ramamoorthy K, Lee J E, Safe S H. Methyl-substituted diindolylmethanes as inhibitors of estrogen-induced growth of T47D cells and mammary tumors in rats. Breast Cancer Res Treat 2001;66:147-57.

What is claimed is:

1. A method for treating atherosclerosis comprising administering to a mammal suffering from atherosclerosis a diindolylmethane analog.

2. The method of claim 1, wherein the diindolylmethane analog is a 1,1-bis(3′-indolyl)-1-(p-substituted phenyl) methane.

3. The method of claim 2, wherein the diindolylmethane analog is 1,1-bis(3′-indolyl)-1-(p-t-butylphenyl) methane.

4. The method of claim 2, wherein the diindolylmethane analog is 1,1-bis(3′-indolyl)-1-(p-biphenyl)methane.

5. The method of claim 1, wherein the mammal is a human.

6. A method for treating endothelial inflammation comprising administering to a mammal suffering from endothelial inflammation a diindolylmethane analog.

7. The method of claim 6, wherein the diindolylmethane analog is a 1,1-bis(3′-indolyl)-1-(p-substituted phenyl) methane.

8. The method of claim 7, wherein the diindolylmethane analog is 1,1-bis(3′-indolyl)-1-(p-t-butylphenyl) methane.

9. The method of claim 7, wherein the diindolylmethane analog is 1,1-bis(3′-indolyl)-1-(p-biphenyl) methane.

10. The method of claim 6, wherein the mammal is a human.

11. A method for inhibiting expression of ICAM-1, MCP-1 or IL-6 comprising, administering a diindolylmethane analog to a mammal.

12. The method of claim 11, wherein the diindolylmethane analog is a 1,1-bis(3′-indolyl)-1-(p-substituted phenyl) methane.

13. The method of claim 12, wherein the diindolylmethane analog is 1,1-bis(3′-indolyl)-1-(p-t-butylphenyl) methane.

14. The method of claim 12, wherein the diindolylmethane analog is 1,1-bis(3′-indolyl)-1-(p-biphenyl) methane.

15. The method of claim 11, wherein the mammal is a human.
